(19) United States
(12) Patent Application Publication
(10) Pub. No.: US 2022/0265278 A1
(43) Pub. Date: Aug. 25, 2022

(54) END EFFECTOR FOR MULTI-FIRE CLIP APPLIER

(71) Applicant: Covidien LP, Mansfield, MA (US)

(72) Inventors: Jaroslaw T. Malkowski, North Port, FL (US); Robert Pedros, Oxford, CT (US); Hanspeter Bayer, Meriden, CT (US)

(21) Appl. No.: 17/677,203

(22) Filed: Feb. 22, 2022

Related U.S. Application Data
(60) Provisional application No. 63/153,830, filed on Feb. 25, 2021.

(51) Int. Cl. A61B 17/128; A61B 17/122

(52) U.S. Cl. CPC A61B 17/1285; A61B 17/122

(57) ABSTRACT

An end effector for a multi-fire clip applier includes a housing that supports a plurality of ligation clips that are arranged in two rows within the housing of the end effector. The end effector includes two pushers and two walking beams that interact with the rows of ligation clips to alternatingly advance a distal-most clip from each of the rows of ligation clips into a jaw assembly of the end effector.

### CROSS-REFERENCE TO RELATED APPLICATION

This application claims the benefit of and priority to U.S. Provisional Patent Application No. 63/153,830, filed Feb. 25, 2021, the entire contents of which is incorporated by reference herein.

### FIELD

This technology is generally related to surgical clip appliers and, more particularly, to an end effector for a multi-fire clip applier.

### BACKGROUND

Endoscopic ligation clip appliers are used to apply ligation clips to body vessels during surgical procedures to occlude or partially occlude the body vessels. These clip appliers are inserted through small diameter cannulas or small incisions in a patient's body to access a surgical site within a body cavity. Performing a surgical procedure endoscopically reduces the amount of trauma inflicted on a patient during a surgical procedure to minimize patient discomfort and reduce patient recovery times.

Surgical clip appliers include single-fire clip appliers and multi-fire clip appliers. In single-fire clip appliers, a single ligation clip is loaded into jaws of the clip applier before each use. Typically, the clip applier is used to withdraw a single clip from a clip package to load the clip into the jaws of the clip applier prior to each use of the clip applier. During an endoscopic procedure in which a single-fire clip applier is used, the clip applier is removed from a body cavity after each use to reload a ligation clip into the clip applier. This process is time consuming and increases the possibility of infection, thus increasing trauma to the patient.

Multi-fire clip appliers include an elongate body that includes a plurality of ligation clips that are sequentially supplied to the jaws of the clip applier to facilitate placement of multiple clips on a body vessel or on body vessels without withdrawing the clip applier from within a body cavity. In some multi-fire clip appliers, the plurality of ligation clips is aligned in tip-to-tail fashion and are fed sequentially into jaws of the clip applier to apply the clips to tissue.

Some endoscopic multi-fire clip appliers include a body portion and an end effector that supports the plurality of clips and can articulate in relation to the body portion. Articulation of the end effector in relation to the body portion provides greater access to tissue within a body cavity of a patient during an endoscopic procedure. Since the plurality of clips is supported in tip to tail fashion, increasing the number of clips within the end effector requires increasing a length of the end effector. Increasing the length of the end effector increases the arc of rotation of the end effector and, thus, limits access of the end effector to tissue.

A continuing need exists in the art for an end effector for a multi-fire articulating clip applier that can support a greater number of clips in a shorter length.

### SUMMARY

This disclosure is directed an end effector for a multi-fire clip applier that includes a housing that supports a plurality of ligation clips that are arranged in two rows within the housing of the end effector. The end effector includes two pushers and two walking beams that interact with the rows of ligation clips to alternatingly advance a distal-most clip from each of the rows of ligation clips into a jaw assembly of the end effector.

Aspects of this disclosure are directed to an end effector including a housing, a plurality of ligation clips, a jaw assembly, and pushers. The housing defines a longitudinal axis and a cavity and has a proximal portion and a distal portion. The plurality of ligation clips are received within the cavity of the housing. Each of the plurality of ligation clips is movable from an open position to a clamped position. The plurality of ligation clips are aligned in first and second side by side rows that extend along the longitudinal axis of the housing. The jaw assembly is supported on the distal portion of the housing. The jaw assembly includes first and second jaws that are movable in relation to each other to move the jaw assembly between open and closed positions. The first and second jaws are configured to sequentially receive each of the plurality of ligation clips from the first and second rows of ligation clips such that movement of the jaw assembly from the open position to the closed position moves the ligation clip received within the jaw assembly from the open position to the clamped position. The pushers are positioned adjacent each of the first and second rows of ligation clips and are movable between retracted and advanced positions to advance the distal-most ligation clips of the first and second rows of ligation clips into the jaw assembly in an alternating fashion.

Other aspects of the disclosure are directed to an end effector including a housing, a plurality of ligation clips, and a jaw assembly. The housing defines a longitudinal axis and a cavity and has a proximal portion and a distal portion. The plurality of ligation clips is received within the cavity of the housing. Each of the plurality of ligation clips is movable from an open position to a clamped position. The plurality of ligation clips is aligned in first and second side by side rows that extend along the longitudinal axis of the housing. The ligation clips in the first row are staggered in relation to the ligation clips in the second row. The jaw assembly is supported on the distal portion of the housing and includes first and second jaws that are movable in relation to each other to move the jaw assembly between open and closed positions. The first and second jaws are configured to sequentially receive each of the plurality of ligation clips from the first and second rows of ligation clips.

In aspects of the disclosure, the first row of ligation clips and the second row of ligation clips are staggered such that one of the first and second rows of ligation clips includes a first distal-most ligation clip.

In some aspects of the disclosure, the pushers each include a transverse driver.

In certain aspects of the disclosure, the transverse driver of the pusher positioned adjacent the first or second row of ligation clips having the first distal-most clip is positioned proximally of the distal-most clip when the pushers are in their retracted positions.

In aspects of the disclosure, each of the pushers includes a resilient stabilizing finger that is positioned to engage the distal-most clip to urge the distal-most clip into the jaw assembly when the pushers are moved from their retracted positions towards their advanced positions.

In some aspects of the disclosure, each of the pushers includes a resilient arm that supports the transverse driver and the stabilizing finger.

In certain aspects of the disclosure, the resilient arm of the pusher positioned adjacent the distal-most clip is deformable as the pusher is moved to its retracted position to allow the transverse driver to move proximally of the distal-most ligation clip.

In aspects of the disclosure, the end effector includes a walking beam coupled to each of the pushers.

In some aspects of the disclosure, the walking beams are movable between retracted and advanced positions in response to movement of the pushers between their retracted and advanced positions to advance the first and second rows of ligation clips within the housing.

In certain aspects of the disclosure, each of the walking beams includes a plurality of resilient fingers that are longitudinally spaced along the walking beam and engage the plurality of ligation clips when the walking beams are in their retracted positions such that movement of the walking beams from their retracted positions towards their advanced positions advances the first and second rows of ligation clips within the housing.

In aspects of the disclosure, each of the pushers includes proximal and distal abutment surfaces and each of the walking beams includes a transverse extension having proximal and distal ends.

In some aspects of the disclosure, the proximal and distal abutment surfaces of the pushers are positioned to engage the proximal and distal ends of the transverse extensions such that movement of the pushers between their advanced and retracted positions causes movement of the walking beams between their advanced and retracted positions.

In certain aspects of the disclosure, the end effector includes jaw link drivers supported on each side of the housing.

In aspects of the disclosure, the jaw link drivers are coupled to the jaw assembly and movable between retracted and advanced positions to move the jaw assembly between the open and closed positions.

In some aspects of the disclosure, each of the jaw link drivers is coupled to the jaw assembly by a linkage that includes first and second links.

In certain aspects of the disclosure, the end effector includes a carriage assembly including a carriage body, a jaw link carriage, and a pusher carriage.

In aspects of the disclosure, the carriage body is received partly within the housing.

In some aspects of the disclosure, the jaw link carriage and the pusher carriage are independently movable along the carriage body between retracted and advanced positions, and the pusher carriage is coupled to the pushers and the jaw link carriage is coupled to the jaw link drivers such that movement of the pusher carriage causes corresponding movement of the pushers and movement of the jaw link carriage causes corresponding movement of the jaw link drivers.

In certain aspects of the disclosure, the end effector includes a first drive cable coupled to the jaw link carriage and a second drive cable coupled to the pusher carriage.

In aspects of the disclosure, the first and second drive cables are movable independently to move the jaw link carriage and the pusher carriage between their advanced and retracted positions.

In some aspects of the disclosure, the carriage body supports first and second drums, and the first drive cable is wrapped about the first drum and the second drive cable is wrapped about the second drum.

In certain aspects of the disclosure, each of the ligation clips includes a first beam, a second beam, and a hinge portion coupling the first beam to the second beam.

In aspects of the disclosure, the first beam includes a first boss and the second beam includes a second boss.

In some aspects of the disclosure, each of the first and second jaws defines channels that receive the first and second bosses of the first distal-most ligation clip when the first distal-most ligation clip is advanced into the jaw assembly.

In certain aspects of the disclosure, the housing includes resilient fingers that extend into the cavity and engage the first bosses of the first beams of the plurality of ligation clips to releasably retain the plurality of ligation clips within the cavity of the housing.

Other aspects of the disclosure are directed to a multi-fire clip applier including an end effector and an endoscopic body portion. The end effector includes a housing, a plurality of ligation clips, a jaw assembly, pushers, jaw link drivers, a carriage assembly, and first and second drive cables. The housing defines a longitudinal axis and a cavity and has a proximal portion and a distal portion. The plurality of ligation clips is received within the cavity of the housing. Each of the plurality of ligation clips is movable from an open position to a clamped position. The plurality of ligation clips are aligned in first and second side by side rows that extend along the longitudinal axis of the housing. The jaw assembly is supported on the distal portion of the housing and includes first and second jaws that are movable in relation to each other to move the jaw assembly between open and closed positions. The first and second jaws are configured to sequentially receive each of the plurality of ligation clips from the first and second rows of ligation clips such that movement of the jaw assembly from the open position to the closed position moves the ligation clip received within the jaw assembly from the open position to the clamped position. The pusher is positioned adjacent each of the first and second rows of ligation clips and are movable between retracted and advanced positions to advance the distal-most ligation clips of the first and second rows of ligation clips into the jaw assembly in an alternating fashion. The jaw link drivers are supported on each side of the housing and are coupled to the jaw assembly and movable between retracted and advanced positions to move the jaw assembly between the open and closed positions. The carriage assembly including a carriage body, a jaw link carriage, a pusher carriage, and first and second drums supported on the carriage body. The carriage body is received partly within the housing. The jaw link carriage and the pusher carriage are independently movable along the carriage body between retracted and advanced positions. The pusher carriage is coupled to the pushers and the jaw link carriage is coupled to the jaw link drivers such that movement of the pusher carriage causes corresponding movement of the pushers and movement of the jaw link carriage causes corresponding movement of the jaw link drivers. The first and second drums are supported on the carriage body. The first drive cable is wrapped about the first drum and the second drive cable is wrapped about the second drum. The first drive cable is coupled to the jaw link carriage and the second drive cable is coupled to the pusher carriage such that the first and second drive cables are movable independently to move the jaw link carriage and the pusher carriage between their advanced and retracted positions. The endoscopic body portion has a distal portion defining a clevis that pivotably supports the carriage body about a pivot axis such that the end effector can pivot in relation to the endoscopic body portion.

In aspects of the disclosure, the clevis of the endoscopic body portion supports third and fourth drums that are positioned on opposite sides of the carriage body within the clevis.

In some aspects of the disclosure, the first drive cable is supported by the first drum and the second drive cable is supported by the second drum.

In certain aspects of the disclosure, the first drive cable includes a first portion positioned on a first side of the first drum and a second portion positioned on a second side of the first drum, and the second drive cable includes a first portion positioned on a first side of the second drum and a second portion positioned on a second side of the second drum.

In aspects of the disclosure, simultaneous proximal movement of the first and second portions of the first drive cable causes the end effector to pivot in relation to the endoscopic body portion in a first direction, and simultaneous proximal movement of the first and second portions of the second drive cable causes the end effector to pivot in relation to the endoscopic body portion in a second opposite direction.

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of an end effector of a multi-fire clip applier according to aspects of the disclosure with a tool assembly in a partially closed position;

FIG. 2 is an exploded view of the end effector shown in FIG. 1;

FIG. 3 is an enlarged view of the indicated area of detail shown in FIG. 2;

FIG. 4 is an enlarged view of the indicated area of detail shown in FIG. 1;

FIG. 5 is an enlarged view of the indicated area of detail shown in FIG. 2;

FIG. 6 is a side perspective view of a carriage assembly including a carriage body, jaw link carriage and pusher carriage of the end effector shown in FIG. 7;

FIG. 7 is a side perspective view of a proximal portion of the end effector shown in FIG. 1 coupled to a distal portion of a body of clip applier;

FIG. 8 is a side perspective view of a portion of the end effector shown in FIG. 1 illustrating a jaw link driver and the carriage assembly;

FIG. 9 is a side perspective view of a distal portion of the end effector shown in FIG. 7 illustrating the pusher assembly and the carriage assembly and a distal portion of a body of a clip applier;

FIG. 10 is an enlarged view of the indicated area of detail shown in FIG. 5 illustrating a walking beam of the walking beam assembly;

FIG. 10A is a side perspective view of a pusher assembly and walking beam assembly of the end effector shown in FIG. 1;

FIG. 11 is a side perspective view of a ligation clip of the end effector shown in FIG. 1 in an open position;

FIG. 12 is a side perspective view of the ligation clip shown in FIG. 11 in a clamped position;

FIG. 13 is a cross-sectional view of a distal portion of the end effector shown in FIG. 1 in a start position with a distal-most clip of a clip stack spaced from a jaw assembly of the end effector;

FIG. 14 is an enlarged view of the indicated area of detail shown in FIG. 13;

FIG. 15 is a cross-sectional view taken along section line 15-15 of FIG. 13;

FIG. 16 is an enlarged view of the indicated area of detail shown in FIG. 5;

FIG. 17 is a cross-sectional view taken along section line 17-17 of FIG. 15;

FIG. 18 is a top view of the end effector shown in FIG. 13 with the outer tube shown in phantom, the pusher assembly in an advanced position, the distal-most clip positioned within the jaw assembly of the end effector, and the jaw assembly in a partially closed position;

FIG. 19 is a side perspective view of the distal portion of the end effector shown in FIG. 18 with the jaw assembly in the partially closed position;

FIG. 19A is a side perspective view of the pusher assembly and walking beam assembly of the end effector shown in FIG. 18 with the pusher assembly in the advanced position;

FIG. 20 is a side perspective view of the distal portion of the end effector shown in FIG. 19 with the jaw link driver in an advanced position, the jaw assembly in an open position, and the distal-most ligation clip in an open position;

FIG. 21 is a side perspective view of the distal portion of the end effector shown in FIG. 20 with the jaw link driver in a retracted position, the jaw assembly in a closed position, and the ligation clip in a clamped position within the jaw assembly;

FIG. 22 is a side perspective view of the distal portion of the end effector shown in FIG. 21 with the jaw link driver in an advanced position, the jaw assembly in an open position, and the ligation clip in a clamped position about tissue and separated from the jaw assembly;

FIG. 23 is a top view of the end effector shown in FIG. 13 with the outer tube removed, the pusher assembly in a retracted position, the distal-most clip spaced from the jaw assembly of the end effector, and the jaw assembly in a partially closed position;

FIG. 23A is a side perspective view of the pusher assembly and walking beam assembly of the end effector shown in FIG. 18 with the pusher assembly in the retracted position;

FIG. 24 is an enlarged view of the indicated area of detail shown in FIG. 23; and

FIG. 25 is a side perspective view with the pusher assembly in its advanced position and a new ligation clip advanced to a position between the first and second jaws.

### DETAILED DESCRIPTION

The disclosed end effector for a multi-fire clip applier will now be described in detail with reference to the drawings in which like reference numerals designate identical or corresponding elements in each of the several views. However, it is to be understood that aspects of the disclosure included herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, and similar terms are used to assist in understanding the description and are not intended to limit the disclosure.

In this description, the term “proximal” is used generally to refer to that portion of the device that is closer to a clinician when the device is used in its customary fashion, while the term “distal” is used generally to refer to that portion of the device that is farther from the clinician when the device is used in its customary fashion. In addition, the term “endoscopic” is used generally to refer to endoscopic, laparoscopic, arthroscopic, and/or any other procedure conducted through a small diameter incision or cannula. Further, the term “clinician” is used generally to refer to medical personnel including doctors, nurses, and support personnel.

The disclosed end effector for a multi-fire clip applier includes a housing that supports a plurality of ligation clips that are arranged in two rows within the housing of the end effector. The end effector includes two pushers and two walking beams that interact with the rows of ligation clips to alternating advance a distal-most clip from each of the rows of ligation clips into a jaw assembly of the end effector.

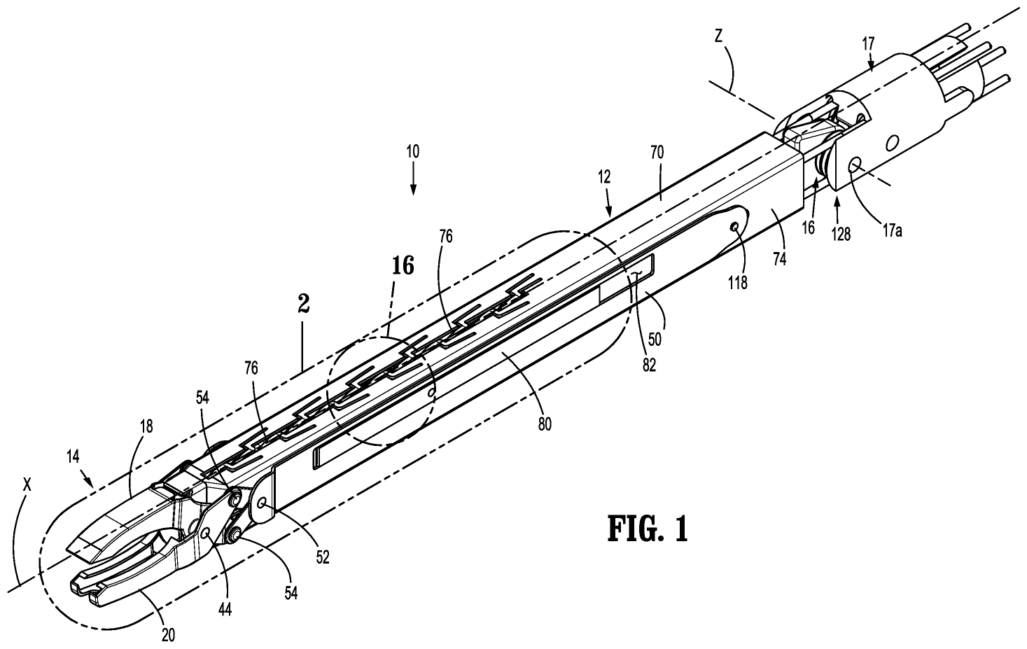

FIG. 1 illustrates an end effector for a multi-fire clip applier shown generally as end effector 10. The end effector 10 defines a longitudinal axis “X” and includes a housing 12, a jaw assembly 14, and a carriage assembly 16. The housing 12 includes a proximal portion that is engaged with the carriage assembly 16 and a distal portion that supports the jaw assembly 14. In aspects of the disclosure, the carriage assembly 16 has a proximal portion that is coupled to a distal portion of an endoscopic body portion 17 of a multi-fire clip applier (not shown) by a pivot member 17a that defines a pivot axis “Z” that is transverse to the longitudinal axis “X” of the end effector. The pivot member 17a facilitates articulation of the end effector 10 in relation to the endoscopic body portion 17 about the pivot axis “Z” between a non-articulated position (FIG. 1) and articulated positions. In the non-articulated position, the longitudinal axis “X” of the end effector is coaxial with a longitudinal axis of the body portion 17. In the articulated positions, the longitudinal axis “X” defines an angle with the longitudinal axis of the body portion 17.

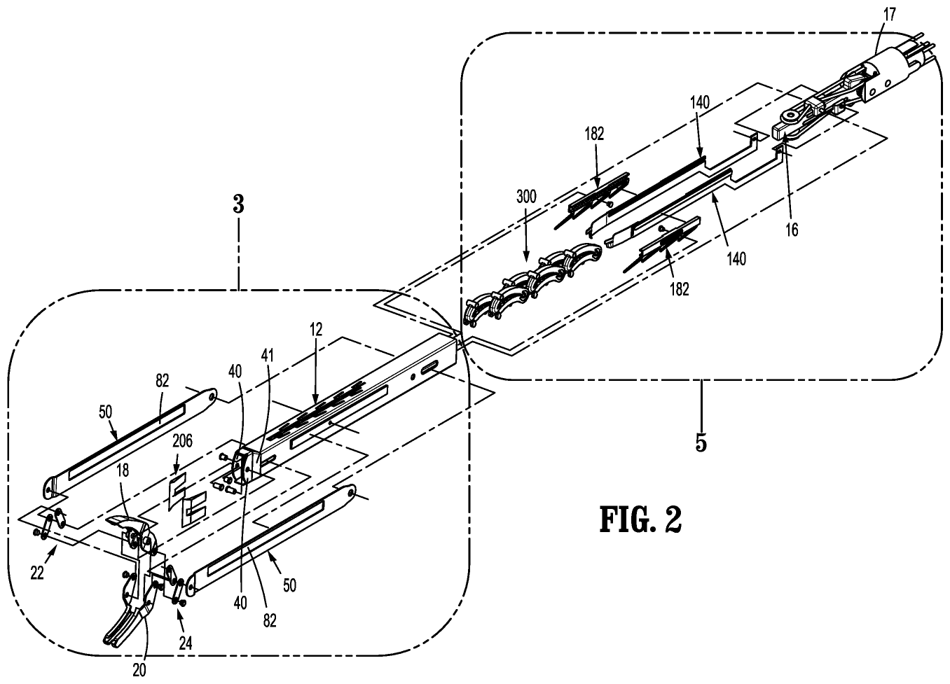

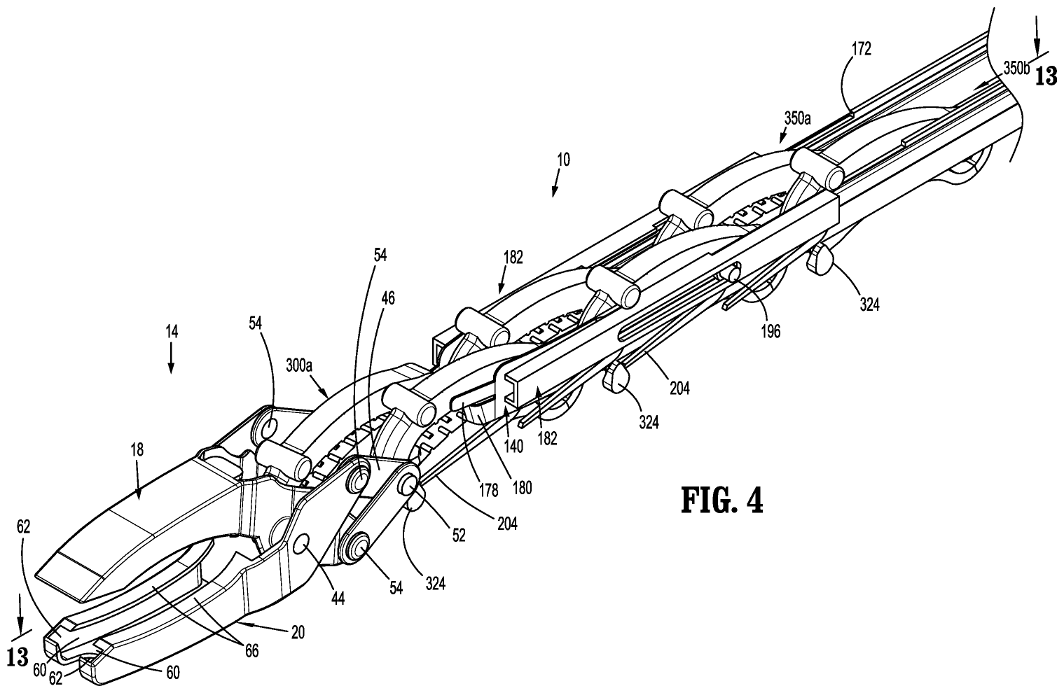

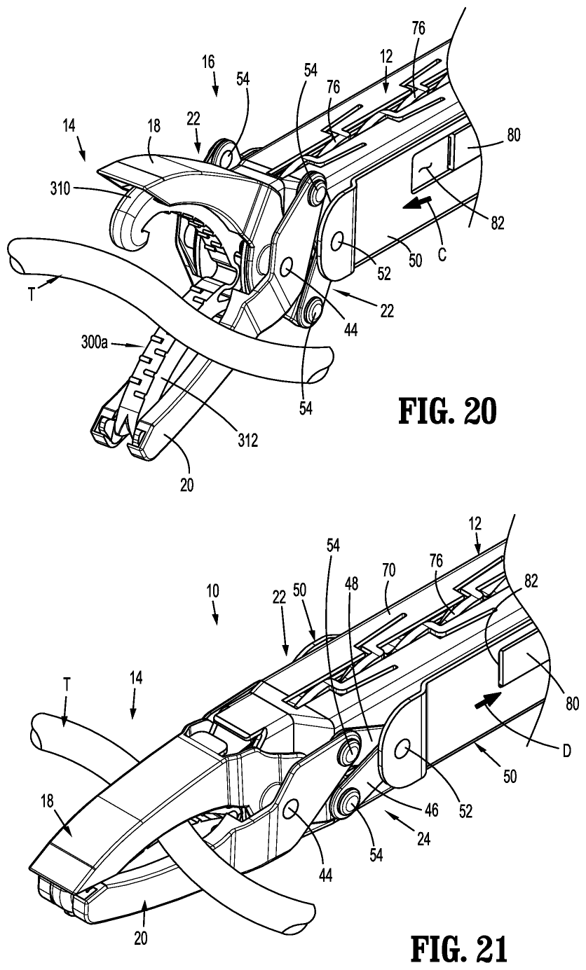

FIGS. 2 to 4 illustrate the jaw assembly 14 which includes a first jaw 18, a second jaw 20, and first and second linkages 22 and 24. Each of the first and second jaws 18 and 20 has a curved configuration and includes a distal clip receiving portion 26 and spaced proximal bracket portions 28. The first jaw 18 includes a centrally located hollow pivot member 30 that extends radially outwardly from a distal portion of each of the bracket portions 28. The second jaw 20 defines an opening 32 in the distal portion of each of the bracket portions 28. The pivot members 30 are received within the openings 32 to couple the first jaw 18 to the second jaw 20 such that the first and second jaws 18 and 20 of the jaw assembly 14 can pivot between an open position (FIG. 20) and a closed position (FIG. 21).

The distal portion of the housing 12 includes spaced brackets 40 that define openings 42 that receive pivot members 44. The pivot members 44 are received within the hollow pivot members 30 of the first jaw 18 to pivotally secure the first and second jaws 18 and 20 to the distal portion of the housing 12. The first and second jaws 18 and 20 are coupled together to the housing 12 by the pivot members 44 in a scissor like fashion, i.e., the first and second jaws 18 and 20 cross at a location adjacent the pivot member 44 such that the clip receiving portion 26 of the first the jaw 18 is positioned on a first side of the second jaw 20 and the bracket portion 28 of the first jaw 18 is positioned on a second opposite side of the second jaw 20.

The first and second linkages 22 and 24 are supported on opposite sides of the housing 12 of the end effector 10 and couple the first and second jaws 18 and 20 to jaw link drivers 50. Each of the first and second linkages 22 and 24 includes a first link 46 and a second link 48. Each of the first and second links 46 and 48 has a proximal end that is pivotally coupled to one of the jaw link drivers 50 by a pivot member 52. More specifically, each of the jaw link drivers 50 defines an opening 50a and each of the first and second links 46 and 48 defines an opening 46a and 48a respectively. The openings 50a, 46a, and 48a receive the pivot member 52 to couple the jaw link drivers 50 to the first and second links 46 and 48 of the first and second linkages 22 and 24. The pivot members 52 extend through longitudinal slots 53 defined in side walls 74 of the housing 12 and are slidable therein as the jaw link drivers 50 are moved between retracted and advanced positions as described below. Each of the first links 46 has a distal end that is coupled to the bracket portion 28 of one of the first and second jaws 18 and 20 by a pivot member 54. Similarly, each of the second links 48 has a distal end that is coupled to the bracket portion 28 of the other of the first and second jaws 18 and 20 by a pivot member 55. As described in further detail below, when the jaw link drivers 50 are moved to their retracted positions, the first and second jaws 18 and 20 are moved to the closed position (FIG. 21) by the first and second linkages 22 and 24, and when the jaw link drivers 50 are moved to their advanced positions, the first and second jaws 18 and 20 are moved to the open position (FIG. 20) by the first and second linkages 22 and 24.

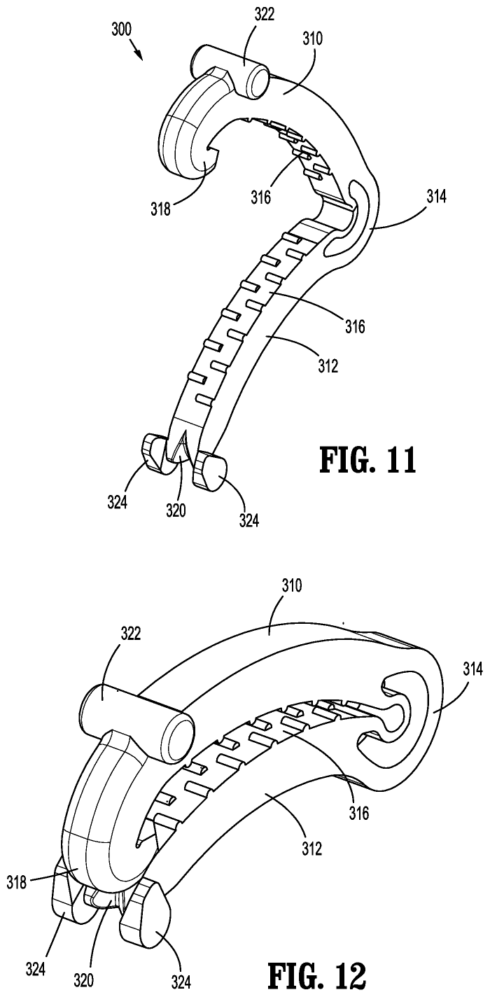

Each of the first and second jaws 18 and 20 defines spaced channels 60 that receive a portion of a ligation clip 300 (FIG. 11). The channels 60 have open distal ends 62 that allow the ligation clip 300 to be removed from the first and second jaws 18 and 20 after the ligation clip 300 is clamped about tissue as described in further detail below. In aspects of the disclosure, the channels 60 are formed by overhangs 66 on the first and second jaws 18 and 20. Alternately, other configurations are envisioned.

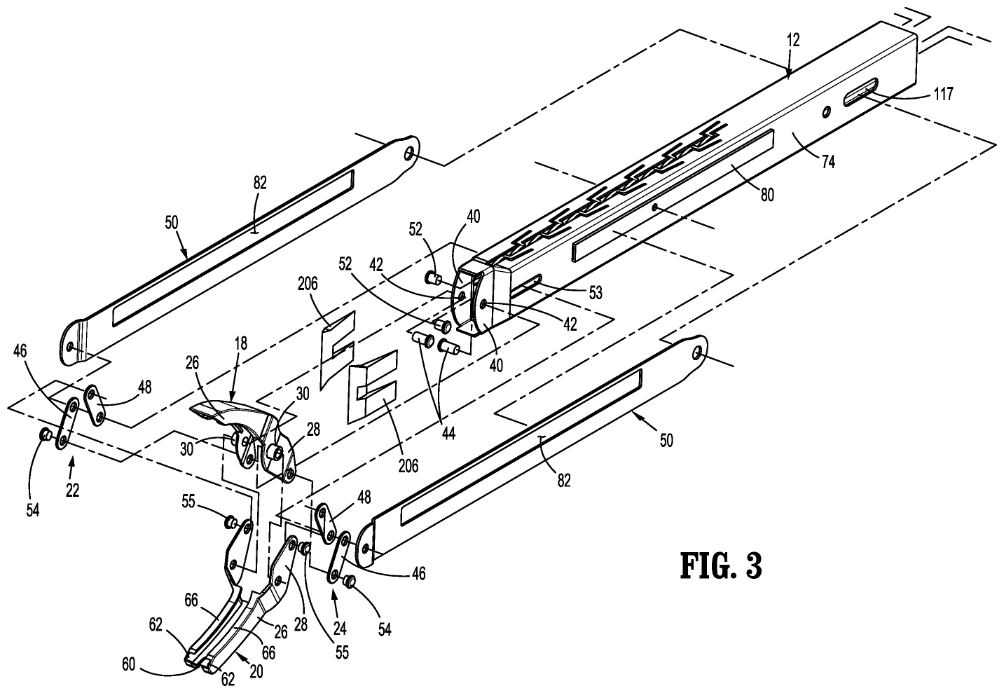

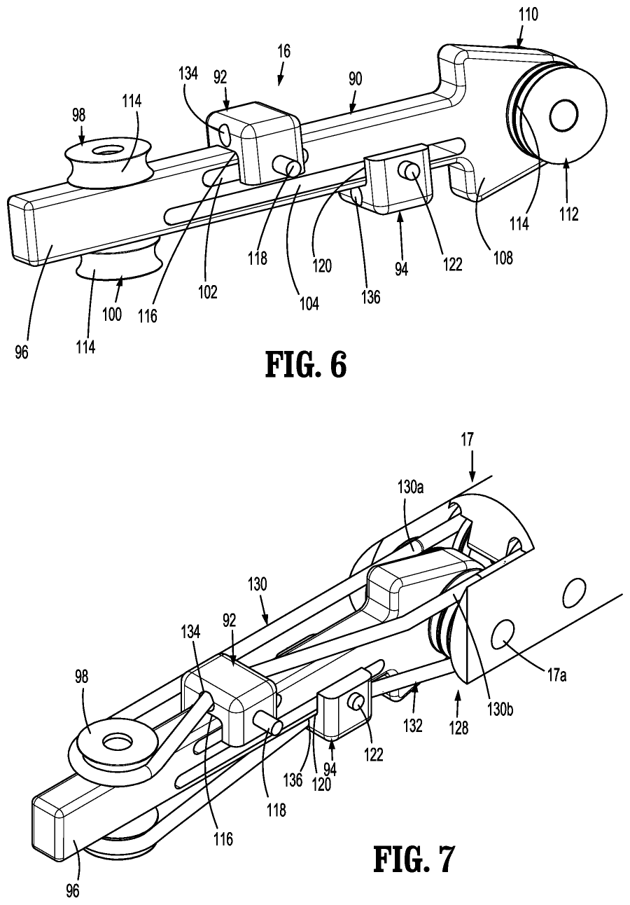

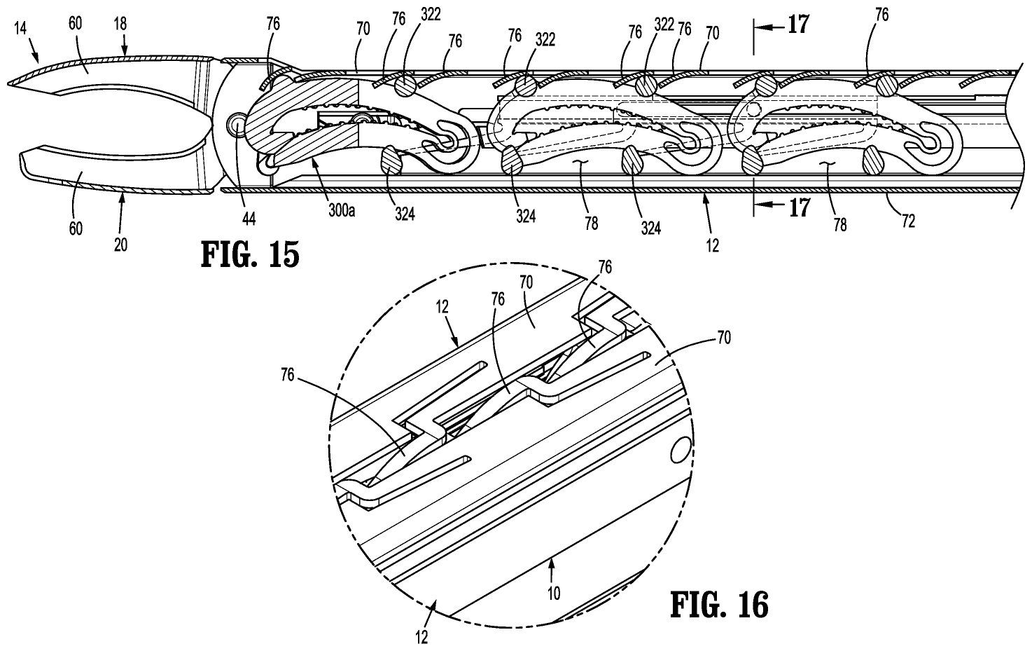

FIGS. 2 and 3 illustrate the housing 12 of the end effector 10. In aspects of the disclosure, the housing 12 is formed from a rigid material such as stainless steel and has a rectangular configuration. The housing 12 includes a distal portion that includes the spaced brackets 40, a converging portion 41, and an open proximal portion that receives a distal portion of the carriage assembly 16 (FIG. 6). The converging portion 41 (FIG. 14) is positioned proximally of the bracket portion 40 of the housing 12 and includes inner walls 41a that converge in the distal direction. The housing 12 has a top wall 70, a bottom wall 72 (FIG. 15), and side walls 74. The distal portion of the housing 12 includes a plurality of inwardly extending resilient fingers 76 that extend from the top wall 70 into a cavity 78 defined by the housing 12. The fingers 76 engage the ligation clips 300 (FIG. 11) to releasably secure the ligation clips 300 at fixed positions within the cavity 78 (FIG. 15) of the housing 12 (FIG. 15). Each of the side walls 74 of the housing 12 includes a raised rail 80 that is received within an elongate slot 82 defined in one of the jaw link drivers 50. The elongate slots 82 have a length that is greater than the lengths of the raised rails 80 to facilitate and guide movement of the jaw link drivers 50 between its retracted and advanced positions. The raised rails 80 have proximal and distal ends that engage the jaw link drivers 50 and act as stop surfaces to define the advanced and retracted positions of the jaw link drivers 50.

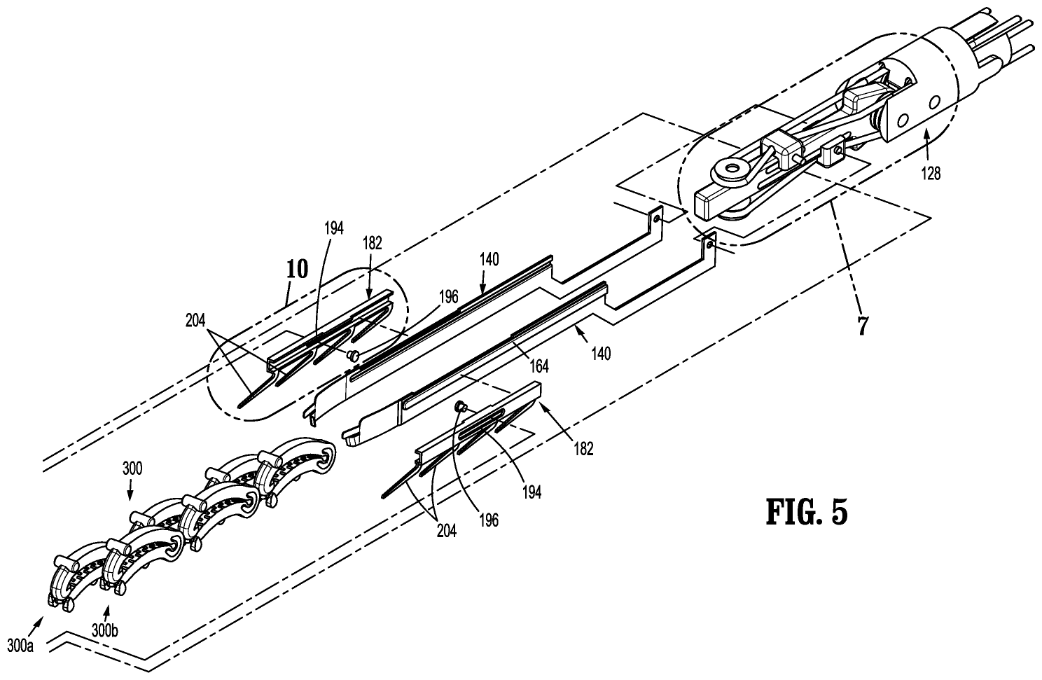

FIGS. 5-7 illustrate the carriage assembly 16 which includes a carriage body 90, a jaw link carriage 92, and a pusher carriage 94. The carriage body 90 has an elongate portion 96 that is received in a proximal portion of the housing 12. The elongate portion 96 of the carriage body 90 supports an upper drum 98 and a lower drum 100 and defines an upper elongate longitudinal slot 102 and a lower elongate longitudinal slot 104. The upper and lower drums 98 and 100 are rotatably secured to the elongate portion 96 of the carriage body 90. In aspects of the disclosure, the elongate portion 96 of the carriage body 90 has a rectangular configuration and the upper and lower drums 98 and 100 are supported on the top and bottom surfaces of the elongate portion 96. The upper and lower elongate slots 102 and 104 extend through the elongate portion 96 between side walls of the carriage body 90. The carriage body 90 also has a proximal portion 108 coupled to, e.g., formed integrally with, the elongate portion 96. The proximal portion 108 supports drums 110 and 112 that are rotatably secured to side walls of the proximal portion 108. Each of the drums 98, 100, 110, and 112 defines a circular groove 114.

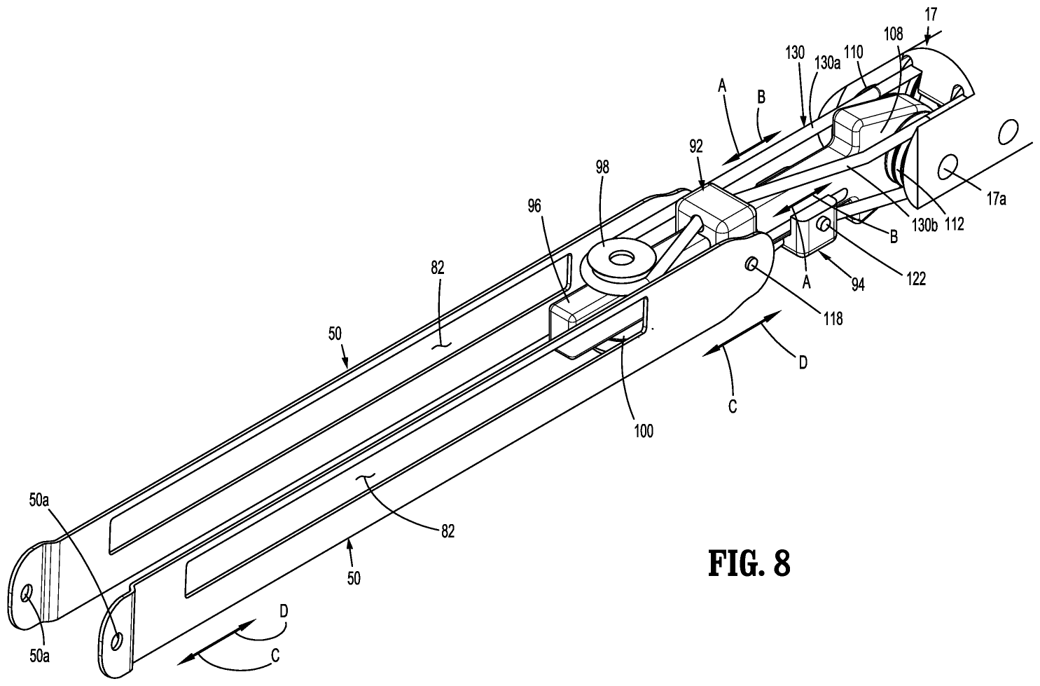

The jaw link carriage 92 is slidably positioned on the top surface of the elongate portion 96 of the carriage body 90 and is movable between retracted and advanced positions. In aspects of the disclosure, the jaw link carriage 92 defines a cutout 116 and supports a shaft or rod 118. The cutout 116 that receives the elongate portion 96 of the carriage body 90 and the shaft 118 extends through the jaw link carriage 92 and through the upper elongate slot 102 defined in the elongate portion 96 of the carriage body 90. The shaft 118 extends from opposite sides of the jaw link carriage 92 through longitudinal slots 117 (FIG. 3) formed in the proximal portion of the housing 12 and is coupled to the jaw link drivers 50 (FIG. 8) as described in further detail below. Receipt of the shaft 118 within the upper elongate slot 102 in the elongate portion 96 of the carriage body 90 and in the longitudinal slot 117 in the housing 12 confines the jaw link carriage 92 to longitudinal movement along the elongate portion 96 of the carriage body 90 between retracted and advanced positions.

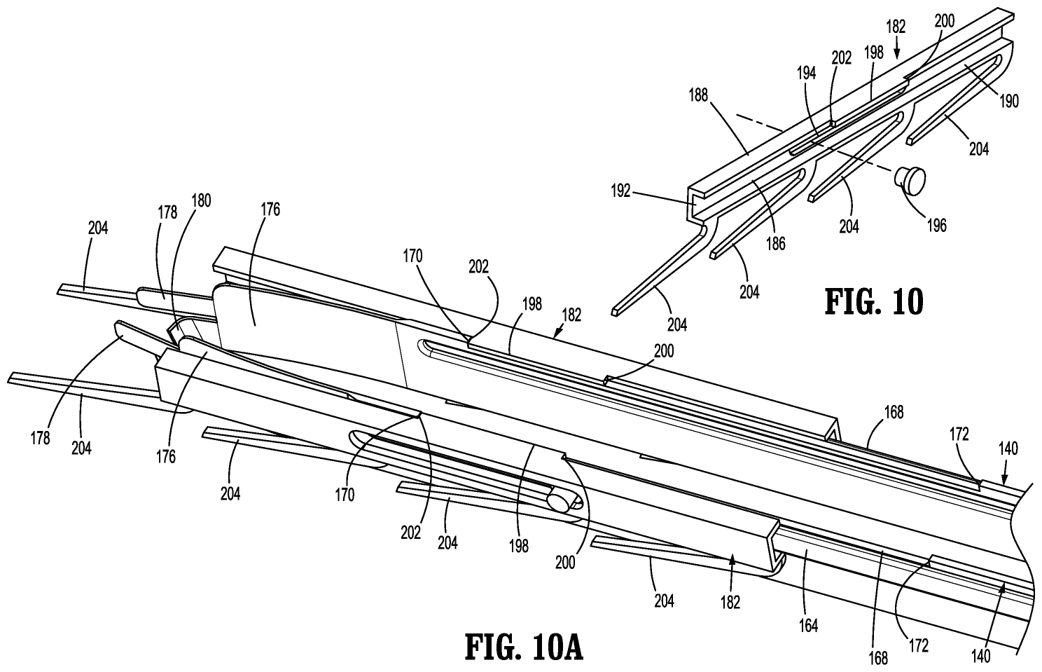

The pusher carriage 94 is slidably positioned along the bottom surface of the elongate portion 96 of the carriage body 90 and is movable between retracted and advanced positions. In aspects of the disclosure, the pusher carriage 94 defines a cutout 120 that receives the elongate portion 96 of the carriage body 90, and a shaft or rod 122 that extends through the pusher carriage 94 and through the lower elongate slot 104 of the elongate portion 96 of the carriage body 90. The shaft 122 extends from opposite sides of the pusher carriage 92 and is coupled to pushers 140 of the end effector 10 (FIG. 10) as described in further detail below. Receipt of the shaft 122 within the lower elongate slot 104 confines the pusher carriage 94 to longitudinal movement along the elongate portion 96 of the carriage body 90 between retracted and advanced positions.

The distal end of the endoscopic body portion 17 of the clip applier (not shown) defines a clevis 128. The proximal portion of the carriage body 90 is received and pivotably secured within the clevis 128 about the pivot member 17a. The pivot member 17a also extends through the drums 110 and 112 to rotatably secure the drums 110 and 112 to the proximal portion 108 of the carriage body 90 within the clevis 128.

The clip applier (not shown) includes first and second drive cables 130 and 132 that extend from an actuator (not shown), e.g., a handle assembly or robotic controller, through the endoscopic body portion 17 (FIG. 7) and into the end effector 10. The first drive cable 130 includes a first portion 130a that extends distally from the endoscopic body portion 17 (FIG. 7) through the groove 114 in the drum 110 and wraps about the drum 98. The first drive cable 130 includes a second portion 130b that is contiguous with the first portion 130a and extends proximally from drum 98, through an opening 134 (FIG. 6) in the jaw link carriage 92, through the groove 114 in the drum 112, and into the endoscopic body portion 17. The second portion 130b is fixedly secured to the jaw link carriage 92 such that longitudinal movement of the first drive cable 130 moves the jaw link carriage 92 between its retracted and advanced positions along the elongate portion 96 of the carriage body 90.

The second drive cable 132 includes a first portion (not shown) that extends distally from the endoscopic body portion 17 (FIG. 7) through the groove 114 in the drum 110 and wraps about the drum 100. The second drive cable 132 includes a second portion 132b that is contiguous with the first portion 132a and extends proximally from drum 100, through an opening 136 (FIG. 6) in the pusher carriage 92, through the groove 114 in the drum 112, and into the endoscopic body portion 17. The second portion 132b is fixedly secured to the pusher carriage 94 such that longitudinal movement of the second drive cable 132 moves the pusher carriage 94 between its retracted and advanced positions along the elongate portion 96 of the carriage body 90.

FIG. 8 illustrates the jaw link drivers 50 coupled to the jaw link carriage 92 by the shaft 118. As described above, when the first drive cable 130 is moved within the endoscopic body portion 17 in the directions indicated by arrows “A” and “B”, the jaw link carriage 92 is moved longitudinally along the elongate portion 96 of the carriage body 90. The proximal portions of the jaw link drivers 50 are coupled to the jaw link carriage 92 such that longitudinal movement of the jaw link carriage 92 moves the jaw link drivers 50 in the directions indicated by arrows C and D between their retracted and advanced positions to move the first and second jaws 18 and 20 (FIG. 1) between the open and closed positions. For example, when the first portion 130a of the first drive cable 130 is moved in the direction of arrow “B”, the second portion 130b of the first drive cable 130 will move in the direction of arrow “A” to move the jaw link carriage 92 in the direction of arrow “A”. When the jaw link carriage 92 is moved in the direction of arrow “A”, the jaw link drivers 50 are moved in the direction of arrows “C” to move the first and second jaws 18 and 20 to the open position (FIG. 20). Similarly, when the second portion 130b of the first drive cable 130 is moved in the direction of arrow “B”, the first portion 130a of the first drive cable 130 will move in the direction of arrow “A” to move the jaw link carriage 92 in the direction of arrow “B”. When the jaw link carriage 92 is moved in the direction of arrow “B”, the jaw link drivers 50 are moved in the direction of arrows “D” to move the first and second jaws 18 and 20 to the closed position (FIG. 21).

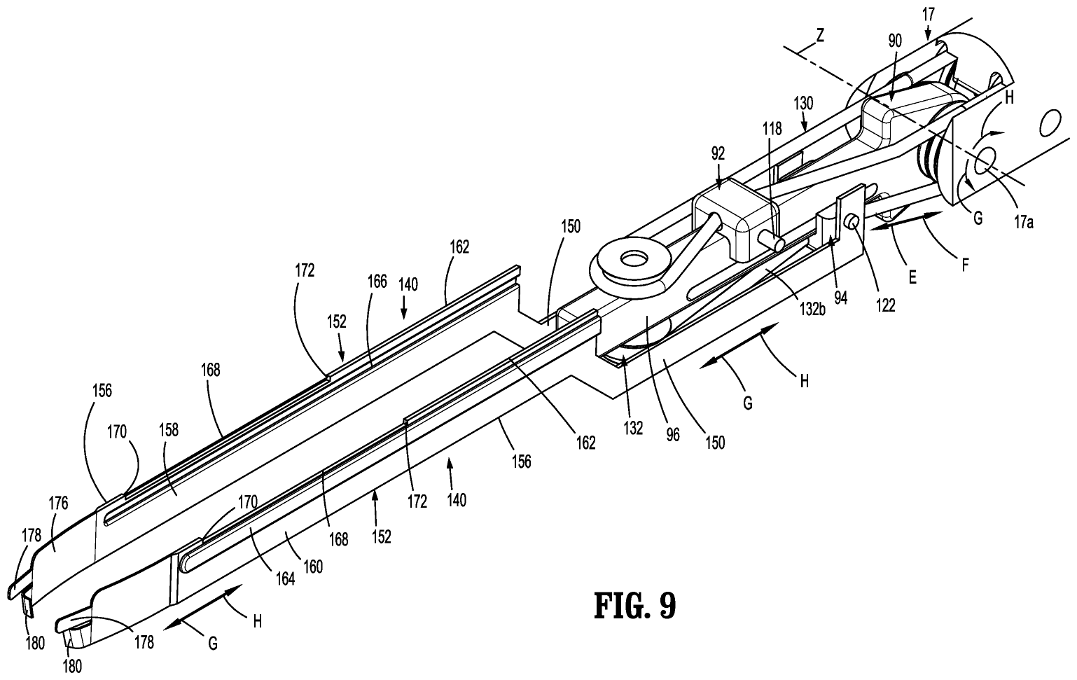

FIG. 9 illustrates the pushers 140 coupled to the pusher carriage 94 by the shaft 122. As described above, when the second drive cable 132 is moved within the endoscopic body portion 17 in the directions indicated by arrows “E” and “F”, the jaw link carriage 92 is moved longitudinally along the elongate portion 96 of the carriage body 90 between its retracted and advanced positions. The proximal portion of the pushers 140 are coupled to the pusher carriage 140 such that longitudinal movement of the pusher carriage 94 moves the pushers 140 in the directions indicated by arrows “G” and “H” between their retracted and advanced positions. For example, when the first portion (not shown) of the second drive cable 132 is moved to move the second portion 132b of the second drive cable 132 in the direction of arrow “E”, the pusher carriage 94 is also moved in the direction of arrow “E” to move the pushers 140 in the direction of arrows “G”. Similarly, when the second portion 132b of the second drive cable 132 is moved in the direction of arrow “F”, the pusher carriage 94 is also moved in the direction of arrow “F” to move the pushers 140 in the direction of arrows “H”.

FIG. 9 illustrates the pushers 140 which are positioned on opposite sides of the carriage body 90. Each of the pushers 140 includes a proximal portion 150 that is coupled to the pusher carriage 94 by the shaft 122 on the pusher carriage 94 and a distal portion 152. The distal portion 152 of each of the pushers 140 includes an elongate beam 156 that includes an inner surface 158, an outer surface 160, and an upper edge 162. The outer surface 160 of each of the beams 156 includes an elongate rib 164 and the inner surface 158 defines a longitudinal groove 166. The upper edge 162 defines an elongate recess 168 that is defined between a distal abutment surface 170 and a proximal abutment surface 172. The distal portion of each of the pushers 140 includes a resilient arm 176, a stabilizing finger 178, and a transverse driver 180. The resilient arms 176 are curved inwardly in the distal direction towards the center of the housing 12 and support the stabilizing fingers 178 and the transverse drivers 180.

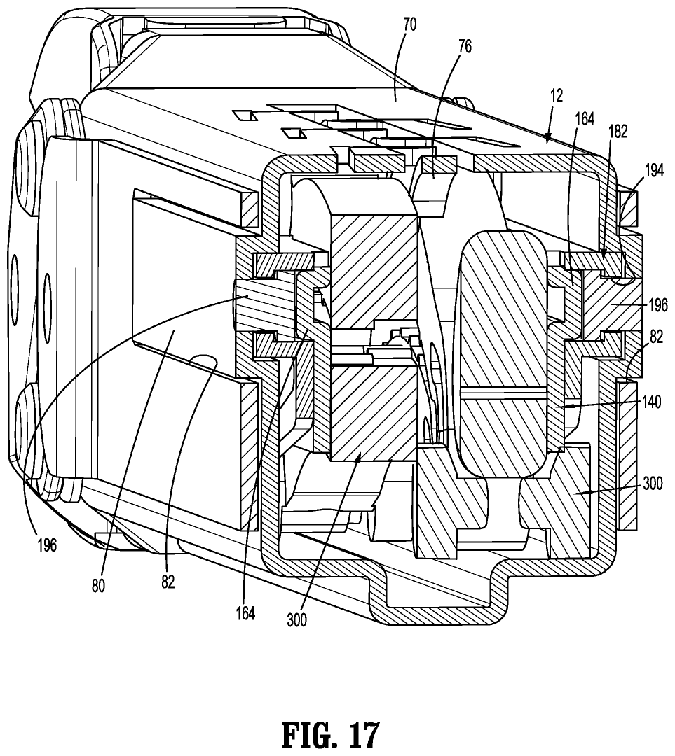

FIGS. 10 and 10A illustrate the interaction between the pushers 140 and walking beams 182. Each of the walking beams 182 includes a C-shaped body 184 that defines a channel 186 (FIG. 10) and includes an upper wall 188, a lower wall 190, and a side wall 192. The side wall 192 defines an elongate slot 194 that receives a pin 196 that is fixedly secured to an inner wall of the housing 12 (FIG. 17). The upper wall 188 of the C-shaped body 184 of each of the walking beams 182 includes a transverse extension 198 that is received within the elongate recess 168 in the upper edge 162 of one of the pushers 140. Each of the transverse extensions 198 includes a proximal end 200 and a distal end 202 that are positioned to engage the proximal and distal abutment surfaces 172 and 170 (FIG. 9), respectively, of the pushers 140 as the pushers are moved between their retracted and advanced positions to translate movement of the pushers 140 into movement of the walking beams 182. The lower wall 190 supports a plurality of spaced resilient fingers 204 that are positioned to engage the ligation clips 300 supported within the housing 12 of the end effector 10 (FIG. 1) as described below.

The walking beams 182 are slidably supported on the outer surfaces 160 of the respective pushers 140. More specifically, the elongate ribs 164 on the pushers 140 are received in the channels 186 defined by the C-shaped body 184 of the walking beams 182 such that the walking beams 182 are slidably supported on the outer surfaces 160 of the pushers 140. When the pushers 140 move between their retracted and advanced positions, the distal and proximal abutment surfaces 170 and 172 on the pushers 140 independently engage the distal and proximal ends 202 and 200 of the transverse extensions 198 on the walking beams 182 to move the walking beams 182 between advanced and retracted positions. More specifically, when the pushers 140 are moved towards their advanced positions, the proximal abutment surface 172 on the pushers 140 engage the proximal ends 200 of the transverse extensions 198 on the walking beams 182 to advance the walking beams 182 with the pushers 140. Similarly, when the pushers 140 are moved towards their retracted positions, the distal abutment surface 170 on the pushers 140 engage the distal ends 202 of the transverse extensions 198 on the walking beams 182 to retract the walking beams 182 with the pushers 140. It is noted that the length of the elongate recesses 168 in the pushers 140 is greater than the length of the transverse extensions 198. As such, the pushers 140 move independently of the walking beams 182 when one of the abutment surfaces 172 and 170 are not engaged with one of the ends 200 and 202 of the transverse extensions 198.

FIGS. 11 and 12 illustrate the ligation clips 300 that include a first beam 310, a second beam 312, and a hinge portion 314. The hinge portion 314 couples the first beam 310 to the second beam 312 and may be in the form of a living hinge. Each of the first and second beams 310 and 312 includes a clamping surface 316. The first beam 310 includes a distal portion that includes a latch 318 and the second beam 312 includes a distal portion that defines a latch receiver 320. Each of the first and second beams 310 and 312 include bosses 322 and 324, respectively, that extend outwardly of the clamping surfaces 316 of the first and second beams 310 and 312. In aspects of the disclosure, the ligation clip 300 is formed from a polymeric material and has a curved configuration. The first and second beams 310 and 312 are movable in relation to each other from an open position (FIG. 11) to a clamped position in which the latch 318 is received within the latch receiver 320 to retain the ligation clip 300 in the clamped position. The ligation clips 300 are aligned in tip-to-tail fashion (FIG. 5) in two side-by-side rows 350a and 350b (FIG. 5) of clips 300. As illustrated, each of the rows 300a and 300b include three ligation clips 300. The ligation clips 300 in each of the rows 300a and 300b are staggered such that the distal-most clip 300a in the row 350a is positioned distally of the distal-most clip 300b by about the length of half of the ligation clip 300.

FIGS. 3 and 14 illustrate wedges 206 that are supported in the end effector 10 to guide the clips 300 into the jaw assembly 14. The wedges 206 are secured to the inner walls 41a of the converging portion 41 of the housing 12. The wedges 41 define a funnel that receives the distal-most ligation clip 300a to direct the distal-most ligation clip 300a into the jaw assembly 14.

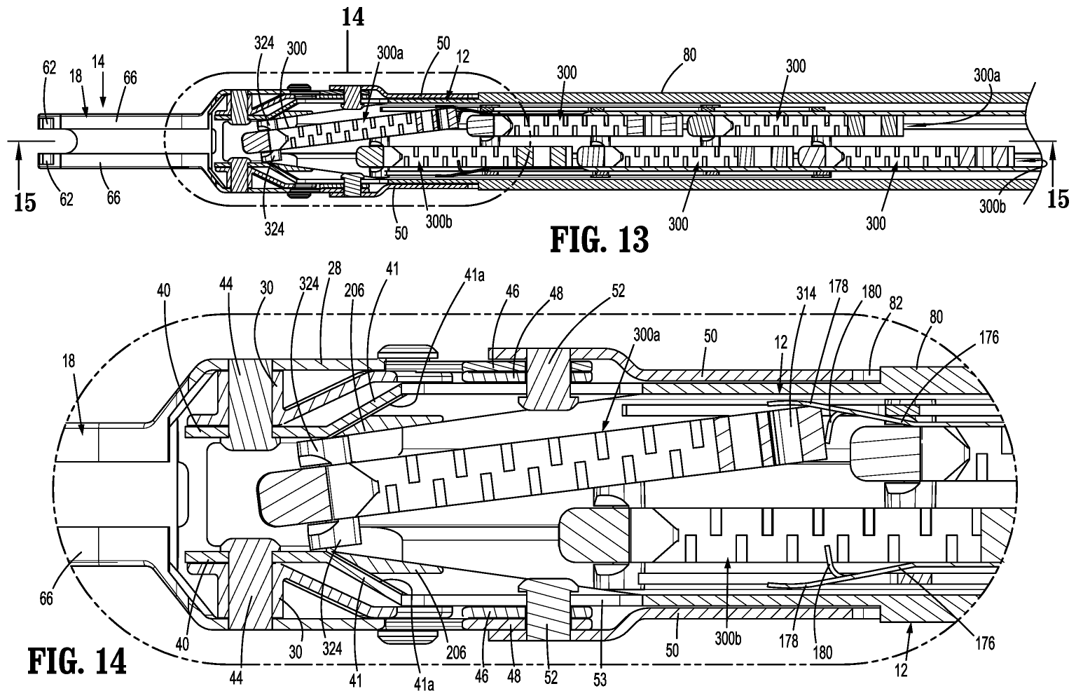

FIGS. 10A, 13-17 illustrate the end effector 10 in a start position prior to firing of the end effector 10. In the start position, the ligation clips 300 are supported within the housing 12 aligned in the rows 350a and 350b (FIG. 13). The bosses 322 on the first beams 310 of the ligation clips 300 are releasably captured between the resilient fingers 76 that extend from the top wall 70 of the housing 12 into the cavity 78 of the housing 12 (FIG. 15) such that the ligation clips 300 in the rows 350a and 350b of ligation clips 300 are retained in staggered relation within the housing 12. In the start position, the distal-most clip 300a in the row 350a of ligation clips 300 is spaced proximally of the jaw assembly 14 (FIG. 14) and the pushers 140 are in a partially advanced position such that the resilient arm 176 of each of the pushers 140 is engaged with the ligation clip 300 positioned proximally of the distal-most clip 300a in the row 350a of ligation clips 300, and the hinge portion 314 (FIG. 13) of the distal-most clip 300a is engaged by the stabilizing finger 178 and transverse driver 180 of the respective pusher 140. When the pushers 140 are in their partially advanced positions, the distal abutment surfaces 170 of the pushers 140 are engaged with the proximal ends 202 of the transverse extensions 198 on the walking beams 182 and the walking beams 182 are in their advanced positions.

In the start position, the jaw link drivers 50 are also in a partially advanced position to position the jaw assembly 14 in a partially closed position. In the partially closed position, the jaws 18 and 20 of the jaw assembly 14 are in a partially approximated position in which the outer surfaces of the jaws 18 and 20 are spaced a distance no greater than the height (or diameter) of the housing 12. In the partially closed position, the jaw assembly 14 can be received within a small diameter cannula to provide access to a body cavity. In the partially closed position, the jaws 18 and 20 are positioned to receive a ligation clip 300 in a partially clamped position. As defined herein, a “partially clamped position” means a position in which the first and second beams 310 and 312 of the ligation clip 300 are partially clamped but the latch 318 of the first beam 310 of the ligation clip 300 is not fully engaged with the latch receiver 320 of the second beam 312 such that the ligation clip 300 can be moved freely between the open and partially clamped positions.

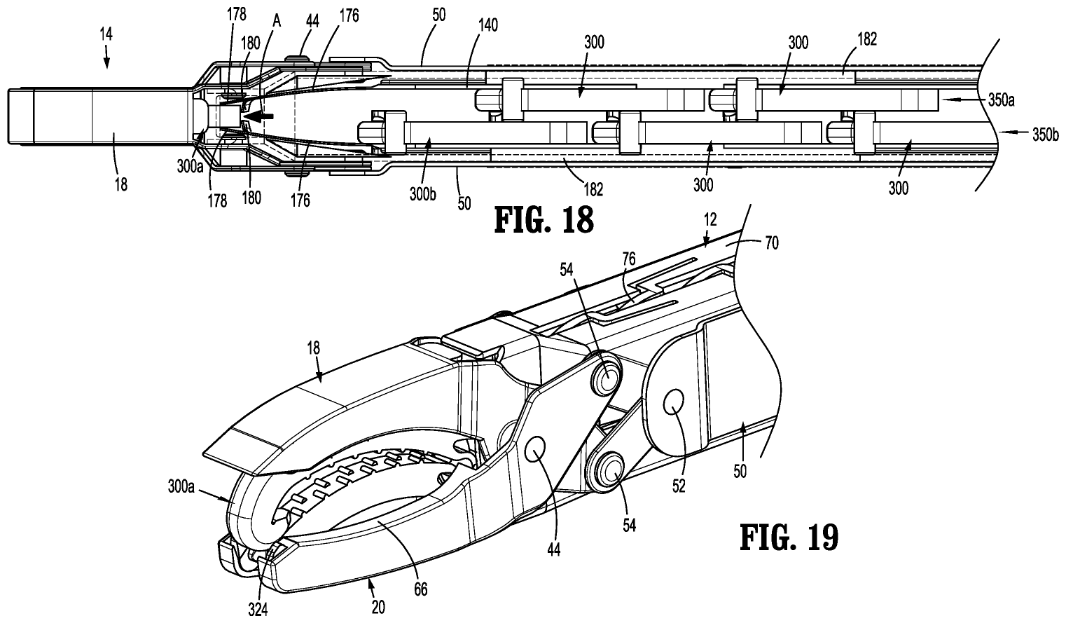

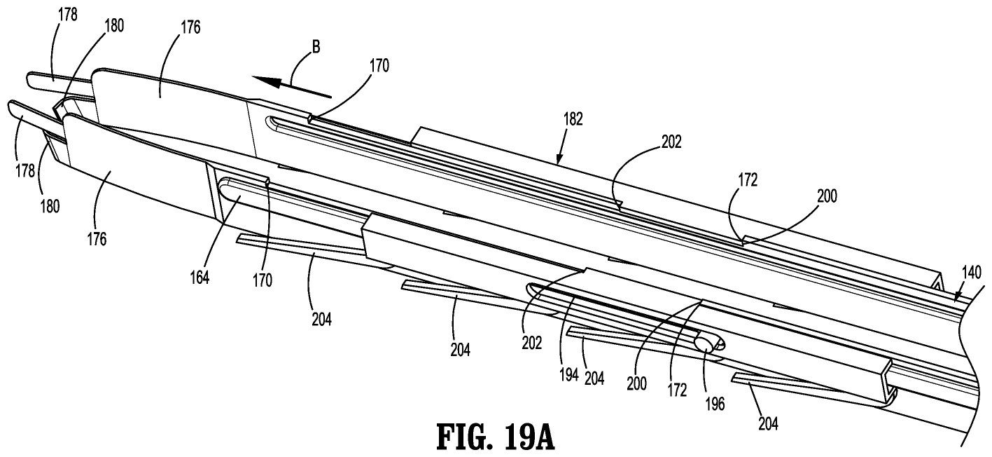

FIGS. 18 to 19A illustrate the end effector 10 as the pushers 140 are moved from the partially advanced position (FIG. 13) to the advanced position in the direction indicated by arrow “A” in FIG. 18. When the pushers 140 are moved from their partially advanced positions to their advanced positions, the transverse driver 180 of the pusher 140 positioned adjacent the row 350a engages and advances the distal-most clip 300a into the jaw assembly 14. As the distal-most clip 300a moves into the jaw assembly 14, the bosses 322 on the first beam 310 of the ligation clip 300a are received in the channels 60 (FIG. 3) of the first jaw 18 of the jaw assembly 14 and the bosses 324 on the second beam 312 of the ligation clip 300a are received in the channels 60 of the second jaw 20 of the jaw assembly 14. As the distal-most clip 300a of the ligation clips 300 is advanced into the jaw assembly 14, the distal-most ligation clip 300a is guided by the wedges 206 from the row 350a of ligation clips 300 towards the central longitudinal axis of the end effector 10 into the jaw assembly 14. As the pushers 140 move towards their advanced positions, the resilient arm 176 of the pusher 140 adjacent the row 350a of clips 300 moves out of engagement with the ligation clip 300 that is positioned proximally of the distal-most clip 300a. This allows the resilient arm 176 of the pusher 140 to flex inwardly to its non-deformed position. As this occurs, the stabilizing finger 178 which is formed on the distal portion of the flexible arm 176 of the pusher 140 moves inwardly to push the distal-most ligation clip 300a inwardly within the housing 12 and align the ligation clip 300a with the jaw assembly 14.

When the pushers 140 move towards their advanced positions from their partially advanced positions (FIG. 10A), the distal abutment surfaces 170 of the pushers 140 move in the direction of arrow “B” in FIG. 19A to a position spaced from the distal end 202 of the transverse extensions 198 of the walking beams 182. Since the abutment surfaces 172 of the pushers 140 are spaced from the proximal end 200 of the transverse extensions 198 of the walking beams 198 as the pushers 140 move from their partially advanced positions towards their advanced positions, the walking beams 182 remain stationary and the pushers 140 move independently of the walking beams 182.

FIG. 20 illustrates the distal portion of the end effector 10 as the jaw assembly 14 is moved from the partially closed position to the open position. When the jaw link drivers 50 are moved in the direction of arrow “C” from their partially advanced positions to their advanced positions, the links 46 and 48 of the first and second linkages 22 and 24 are moved to expanded positions to move the first and second jaws 18 and 20 to the open position. The jaw assembly 14 is moved to the open position to facilitate placement of tissue between the first and second jaws 18 and 20.

FIG. 21 illustrates the distal portion of the end effector 10 as the jaw link drivers 50 are moved in the direction of arrow “D” to move the jaw assembly 14 to the closed position and move the distal-most ligation clip 300a to the clamped position. In the clamped position, the first beam 310 of the distal-most ligation clip 300a is moved into juxtaposed alignment with the second beam 312 such that the latch 318 of the first beam 310 lockingly engages the latch receiver 320 to retain the ligation clip 300a in the clamped position about tissue “T”. When the jaw assembly 14 is moved to the closed position, the pushers 140 remain in their advanced positions and the walking beams 182 remain in their starting positions.

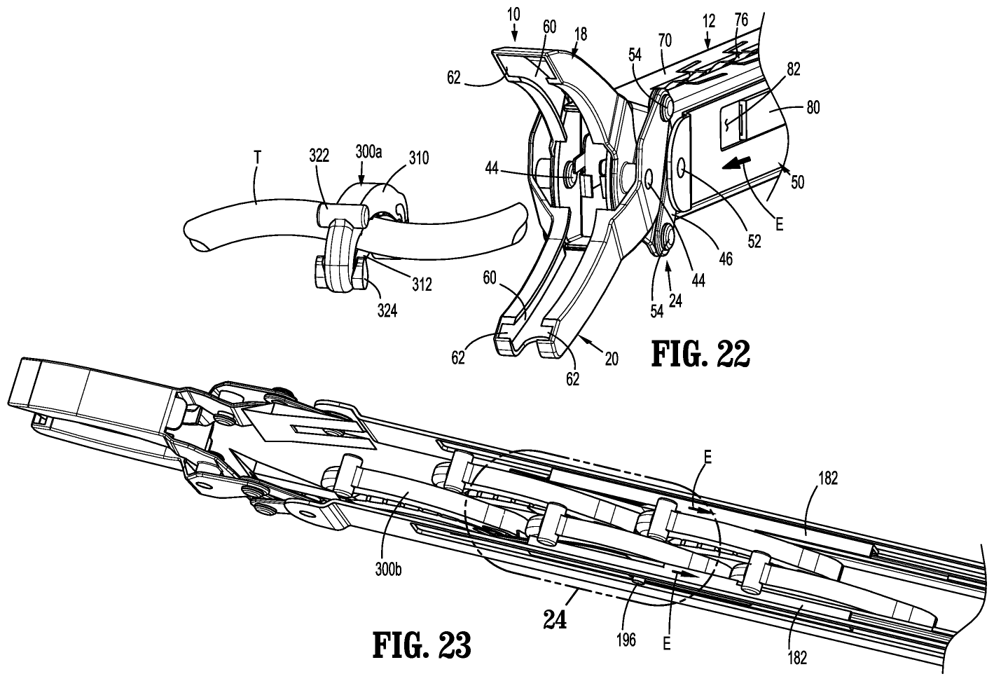

FIG. 22 illustrates the distal portion of the end effector 10 as the jaw link drivers 50 are moved in the direction indicated by arrows “E” to move the jaw assembly 14 back to the open position and release the ligation clip 300a from between the first and second jaws 18 and 20. As described above, when the jaw assembly 14 moves from the closed position to the open position, the bosses 322 and 324 of the first and second beams 310 and 312 of the ligation clip 300a exit the channels 60 in the first and second jaws 18 and 20, respectively through the open distal ends 62 of the channels 60.

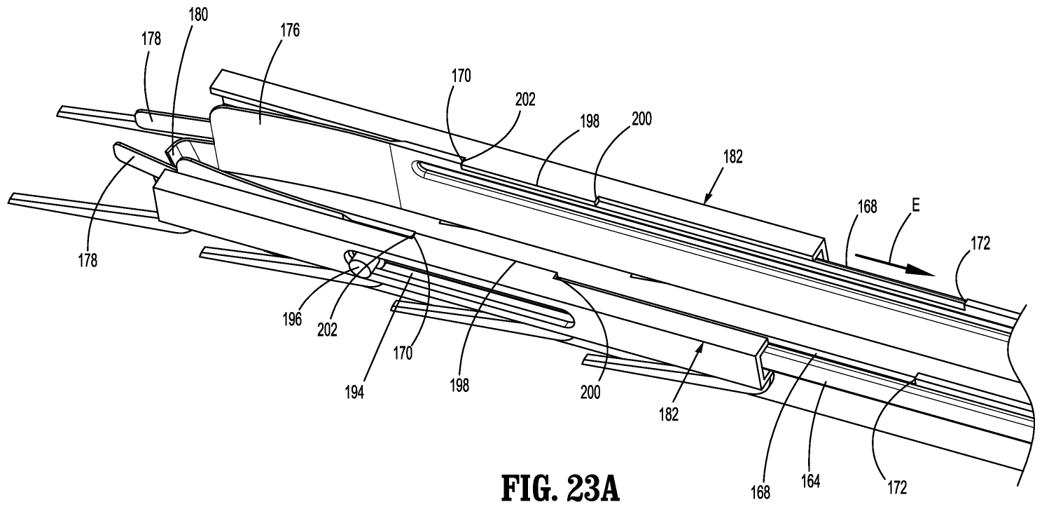

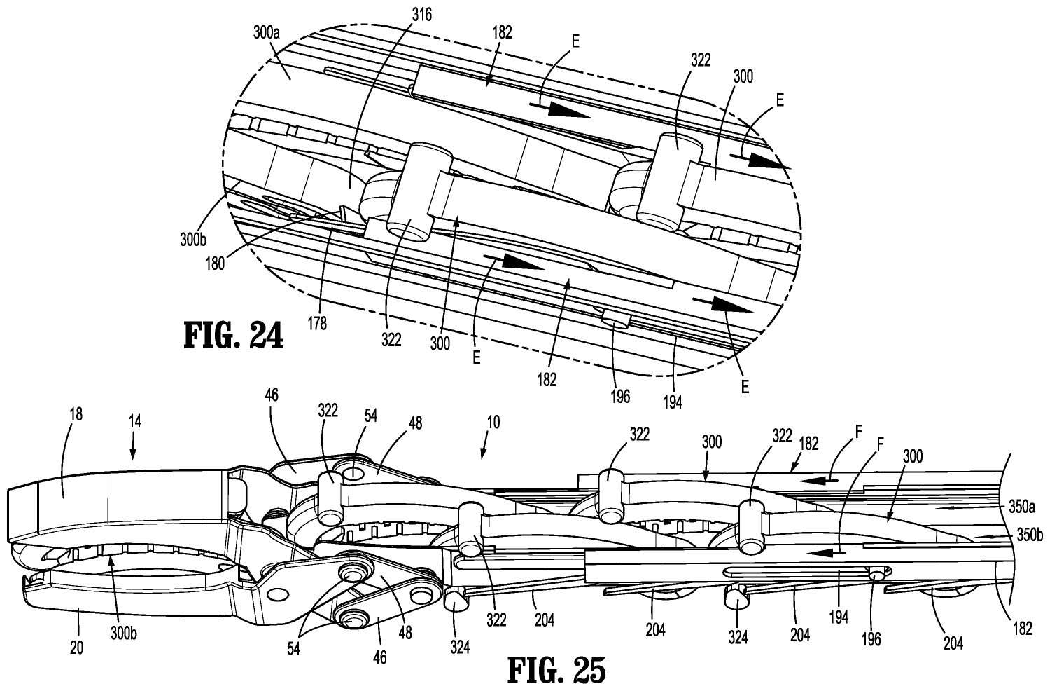

FIGS. 23 and 23A illustrate the distal portion of the end effector 10 when the pushers 140 are moved in the direction of arrows “E” from their advanced positions to their retracted positions. As the pushers 140 move towards their retracted positions, the pushers 140 will move independently of the walking beams 182 until the distal abutment surfaces 170 on the pushers 140 engage the distal ends 202 of the transverse extensions 198 on the walking beams 182. When this occurs, the walking beams 182 will move with the pushers 140 to their retracted positions. As the pushers 140 move to their retracted positions, the resilient arm 176 of the pusher 140 adjacent the row 350b of ligation clips 300 with the distal-most clip, now ligation clip 300b, engages the ligation clip 300 positioned proximally of the distal-most clip 300b and is biased outwardly to move the transverse driver 180 to allow the transverse drive 180 of the pusher 140 to pass by the distal-most clip 300b to a position proximally of the hinge portion 316 of the distal-most clip 300b. When the walking beams 182 move to their retracted position, the resilient fingers 204 on the lower wall 190 of each of the walking beams 182 moves to a position proximally of the bosses 324 on the second beam 312 of the ligation clips 300 in each of the rows 350a and 350b. It is noted that since the clips in the rows 350a and 350b are staggered, the stroke of the walking beams 182 is such that the walking beams 182 will advance the rows 350a and 350b a half of a clip length. As such, only every other resilient finger 204 of each of the walking beams 182 will engage a boss 324 of a ligation clip 300 during each stroke of the walking beams 182 when the end effector 10 is fully loaded with ligation clips 300.

FIG. 25 illustrates the distal portion of the end effector 10 when the pushers 140 are moved from their retracted positions to their advanced positions. As the pushers 140 are advanced, the pushers 140 initially move independently of the walking beams 182 in the direction of arrows “F” to move the distal-most clip 300b from the row 350b of ligation clips 300 into the jaw assembly 14. As described above regarding clip 300a (FIG. 19), the distal-most clip 300b is engaged by the transverse driver 180 of the pusher 140 positioned adjacent the row 350b of ligation clips 300 and advanced into the jaw assembly 14. As the distal-most clip 300b moves into the jaw assembly 14, the bosses 322 on the first beam 310 of the ligation clip 300b are received in the channels 60 (FIG. 3) of the first jaw 18 of the jaw assembly 14 and the bosses 324 on the second beam 312 of the ligation clip 300b are received in the channels 60 of the second jaw 20 of the jaw assembly 14. As the distal-most clip 300b of the ligation clips 300 is advanced into the jaw assembly 14, the distal-most ligation clip 300b is guided by the wedges 206 from the row 350b of ligation clips 300 towards the central longitudinal axis of the end effector 10 into the jaw assembly 14. As the pushers 140 move towards their advanced positions, the resilient arm 176 of the pusher 140 move out of engagement with the ligation clip 300 proximally of the distal-most clip 300b. This allows the resilient arm 176 of the pusher 140 to flex inwardly to its non-deformed position. As this occurs, the stabilizing finger 178 which is formed on the distal portion of the pusher 140 pushes the distal-most ligation clip 300b inwardly within the housing 12 into alignment with the jaw assembly 14.

At some point during movement of the pushers 140, the proximal ends 200 of the transverse extension 198 is engaged by the proximal abutment surface 172 on the pusher 140 to move the walking beams 182 towards their advanced positions. As the walking beams 182 are moved towards their advanced positions, the fingers 204 move the rows 350a and 350b of ligation clips 300 a half a clip length within the housing 12. When the rows 350a and 350b are advanced a half a clip length, the bosses 322 of the ligation clips 300 in each of the rows 350a and 350b are received between of the resilient fingers 76 (FIG. 15) of the housing 12 to releasably retain the ligation clips 300 within the housing 12 of the end effector 10.

In aspects of the disclosure, the first and second drive cables 130 and 132 can be operated to articulate the end effector 10 about the pivot axis “Z” (FIG. 9). More specifically, if the first and second portions 130a and 130b of the first drive cable 130 are retracted simultaneously the end effector 10 will articulate about the pivot axis “Z” in the direction of arrows “G”. Similarly, if the first portion (not shown) and second portion 132b of the second drive cable 132 are retracted simultaneously the end effector 10 will articulate about the pivot axis “Z” in the direction of arrows “H”.

The steps summarized above are repeated to clamp each of the ligation clips 300 onto tissue “T” and reload the jaw assembly 14 with the next distal-most clip. By providing side by side rows 350a and 350b of ligation clips 300, the length of the end effector 12 for holding a specified number of ligation clips 300 can be minimized, i.e., providing two rows of clips as described above as compared to a single row of clips allows the length of the end effector 10 to be reduced by the (N−1)P, where N is the number of clips in each row of clips and P is the length of each of the clips. This allows greater endoscopic access to tissue when the end effector 10 is articulated within a body cavity during a surgical procedure.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. An end effector comprising:
a housing defining a longitudinal axis and a cavity and having a proximal portion and a distal portion;
a plurality of ligation clips received within the cavity of the housing, each of the plurality of ligation clips being movable from an open position to a clamped position, the plurality of ligation clips aligned in first and second side by side rows that extend along the longitudinal axis of the housing;
a jaw assembly supported on the distal portion of the housing, the jaw assembly including first and second jaws that are movable in relation to each other to move the jaw assembly between open and closed positions, the first and second jaws configured to sequentially receive each of the plurality of ligation clips from the first and second rows of ligation clips, wherein movement of the jaw assembly from the open position to the closed position moves the ligation clip received within the jaw assembly from the open position to the clamped position; and
a pusher positioned adjacent each of the first and second rows of ligation clips, the pushers movable between retracted and advanced positions to advance the distal-most ligation clips of the first and second rows of ligation clips into the jaw assembly in an alternating fashion.

2. The end effector of claim 1, wherein the first row of ligation clips and the second row of ligation clips are staggered such that one of the first and second rows of ligation clips includes a first distal-most ligation clip.

3. The end effector of claim 1, wherein the pushers each include a transverse driver, the transverse driver of the pusher positioned adjacent the first or second row of ligation clips having the first distal-most clip being positioned proximally of the first distal-most clip when the pushers are in their retracted positions.

4. The end effector of claim 3, wherein each of the pushers including a resilient stabilizing finger that is positioned to engage the first distal-most clip to urge the first distal-most clip into the jaw assembly when the pushers are moved from their retracted positions towards their advanced positions.

5. The end effector of claim 4, wherein each of the pushers includes a resilient arm that supports the transverse driver and the stabilizing finger, the resilient arm of the pusher positioned adjacent the first distal-most clip being deformable as the pusher is moved to the retracted position to allow the transverse driver to move proximally of the first distal-most ligation clip.

6. The end effector of claim 1, a walking beam coupled to each of the pushers, the walking beams being movable between retracted and advanced positions in response to movement of the pushers between their retracted and advanced positions to advance the first and second rows of ligation clips within the housing.

7. The end effector of claim 6, wherein each of the walking beams includes a plurality of resilient fingers that are longitudinally spaced along the walking beam, the resilient fingers engaging the plurality of ligation clips when the walking beams are in their retracted positions such that movement of the walking beams from their retracted positions towards their advanced positions advances the first and second rows of ligation clips within the housing.

8. The end effector of claim 8, wherein each of the pushers includes proximal and distal abutment surfaces and each of the walking beams includes a transverse extension having proximal and distal ends, the proximal and distal abutment surfaces of the pushers positioned to engage the proximal and distal ends of the transverse extensions such that movement of the pushers between their advanced and retracted positions causes movement of the walking beams between their advanced and retracted positions.

9. The end effector of claim 1, further including jaw link drivers supported on each side of the housing, the jaw link drivers coupled to the jaw assembly and movable between retracted and advanced positions to move the jaw assembly between the open and closed positions.

10. The end effector of claim 9, wherein each of the jaw link drivers is coupled to the jaw assembly by a linkage that includes first and second links.

11. The end effector of claim 10, further including a carriage assembly including a carriage body, a jaw link carriage, and a pusher carriage, the carriage body being received partly within the housing, the jaw link carriage and the pusher carriage being independently movable along the carriage body between retracted and advanced positions, the pusher carriage coupled to the pushers and the jaw link carriage coupled to the jaw link drivers such that movement of the pusher carriage causes corresponding movement of the pushers and movement of the jaw link carriage causes corresponding movement of the jaw link drivers.

12. The end effector of claim 11, further including a first drive cable coupled to the jaw link carriage and a second drive cable coupled to the pusher carriage, the first and second drive cables movable independently to move the jaw link carriage and the pusher carriage between their advanced and retracted positions.

13. The end effector of claim 11, wherein the carriage body supports first and second drums, the first drive cable wrapped about the first drum and the second drive cable wrapped about the second drum.

14. The end effector of claim 1, wherein each of the ligation clips includes a first beam, a second beam, and a hinge portion coupling the first beam to the second beam, the first beam including a first boss and the second beam including a second boss.

15. The end effector of claim 1, wherein each of the first and second jaws defines channels that receive the first and second bosses of the first distal-most ligation clip when the first distal-most ligation clip is advanced into the jaw assembly.

16. The end effector of claim 14, wherein the housing includes resilient fingers that extend into the cavity and engage the first bosses of the first beams of the plurality of ligation clips to releasably retain the plurality of ligation clips within the cavity of the housing.

17. A multi-fire clip applier comprising:
an end effector including:
a housing defining a longitudinal axis and a cavity and having proximal portion and distal portions;
a plurality of ligation clips received within the cavity of the housing, each of the plurality of ligation clips being movable from an open position to a closed position, the plurality of ligation clips aligned in first and second side by side rows that extend along the longitudinal axis of the housing;
a jaw assembly supported on the distal portion of the housing, the jaw assembly including first and second jaws that are movable in relation to each other to move the jaw assembly between open and closed positions, the first and second jaws configured to sequentially receive each of the plurality of ligation clips from the first and second rows of ligation clips, wherein movement of the jaw assembly from the open position to the closed position moves the ligation clip received within the jaw assembly from the open position to the clamped position; and
a pusher positioned adjacent each of the first and second rows of ligation clips, the pushers movable between retracted and advanced positions to advance the distal-most ligation clips of the first and second rows of ligation clips into the jaw assembly in an alternating fashion;
jaw link drivers supported on each side of the housing, the jaw link drivers coupled to the jaw assembly and movable between retracted and advanced positions to move the jaw assembly between the open and closed positions;
a carriage assembly including a carriage body, a jaw link carriage, a pusher carriage, and first and second drums supported on the carriage body, the carriage body received partly within the housing, the jaw link carriage and the pusher carriage being independently movable along the carriage body between retracted and advanced positions, the pusher carriage coupled to the pushers and the jaw link carriage coupled to the jaw link drivers such that movement of the pusher carriage causes corresponding movement of the pushers and movement of the jaw link carriage causes corresponding movement of the jaw link drivers, the first drive cable wrapped about the first drum and the second drive cable wrapped about the second drum; and
a first drive cable coupled to the jaw link carriage and a second drive cable coupled to the pusher carriage, the first and second drive cables movable independently to move the jaw link carriage and the pusher carriage between their advanced and retracted positions; and
an endoscopic body portion having a distal portion defining a clevis, the clevis pivotably supporting the carriage body about a pivot axis such that the end effector can pivot in relation to the endoscopic body portion.

18. The multi-fire clip applier of claim 17, wherein the clevis of the endoscopic body portion supports third and fourth drums, the third and fourth drums positioned on opposite side of the carriage body within the clevis, the first drive cable supported by the first drum and the second drive cable supported by the second drum.

19. The multi-fire clip applier of claim 18, wherein the first drive cable includes a first portion positioned on a first side of the first drum and a second portion positioned on a second side of the first drum, and the second drive cable includes a first portion positioned on a first side of the second drum and a second portion positioned on a second side of the second drum, wherein simultaneous proximal movement of the first and second portions of the first drive cable causes the end effector to pivot in relation to the endoscopic body portion in a first direction, and simultaneous proximal movement of the first and second portions of the second drive cable causes the end effector to pivot in relation to the endoscopic body portion in a second opposite direction.

20. An end effector comprising:
a housing defining a longitudinal axis and a cavity and having a proximal portion and a distal portion;
a plurality of ligation clips received within the cavity of the housing, each of the plurality of ligation clips being movable from an open position to a clamped position, the plurality of ligation clips aligned in first and second side by side rows that extend along the longitudinal axis of the housing, wherein the ligation clips in the first row are staggered in relation to the ligation clips in the second row; and
a jaw assembly supported on the distal portion of the housing, the jaw assembly including first and second jaws that are movable in relation to each other to move the jaw assembly between open and closed positions, the first and second jaws configured to sequentially receive each of the plurality of ligation clips from the first and second rows of ligation clips.
